(19) United States
(12) Patent Application Publication
(10) Pub. No.: US 2018/0098754 A1
(43) Pub. Date: Apr. 12, 2018

(54) IMAGE COMPENSATION SYSTEM FOR COMPENSATING ECHO SIGNALS AND METHOD THEREOF

(71) Applicant: NATIONAL TAIWAN UNIVERSITY, Taipei (TW)

(72) Inventors: Pai-Chi LI, Taipei (TW); U-Wai LOK, Taipei (TW)

(21) Appl. No.: 15/445,360

(22) Filed: Feb. 28, 2017

(30) Foreign Application Priority Data
Oct. 6, 2016 (TW) 105132392

(51) Int. Cl. A61B 8/08; A61B 8/14

(52) U.S. Cl. CPC A61B 8/5269; A61B 8/14

(57) ABSTRACT

An image compensation system and method which provided to compensate echo signals acquired from an ultrasound probe which are provided in the present invention. M channels of the ultrasound probe are divided into m groups where each group consists of n channels. For each group, image compensation system takes the average value from n channel data to achieve an average-signal strength echo signal and then computes n error values according to the average-signal strength echo signal and n echo signals within a group. The image compensation system determines n plus and minus sign operators according to the positivity or negativity of the n error values. The image compensation system then calculates a mean absolute error value according to the n error values. Thereafter, n compensated echo signals of the m groups according to the average-signal echo strength, plus and minus sign operators, and the mean absolute error value are digitized.

This application claims the benefit of Taiwan Patent Application Serial No. 105132392, filed Oct. 6, 2016, the subject matter of which is incorporated herein by reference.

### BACKGROUND OF INVENTION

### 1. Field of the Invention

The present invention is related to an image compensation system and a method thereof, and more particularly is related to the image compensation system and the method thereof which compensate echo signals by using plus-or-minus operators, arithmetic mean, and average signal strength value.

### 2. Description of the Prior Art

Ultrasound imaging has been widely applied to medical diagnosis. Compared to other clinical medical imaging modalities such as X-ray, CT, MRI and nuclear imaging systems, ultrasound imaging is characterized as cost effective, non-invasive, free of ionizing radiation, real-time, portable, capability of flow detection, etc. Hence, ultrasound imaging has been widely utilized to assist clinical diagnosis. Ultrasound imaging is based on reflection and backscattering. Specifically, a probe is required for radiating a sound wave into a human body. The interaction between sound wave and the tissues inside the human body produces echoes that are detected by the probe and images are reconstructed by the system based on the received echoes.

The imaging process of ultrasound imaging needs a calculation circuit as shown in FIG. 1. FIG. 1 is a block diagram showing a first conventional ultrasound imaging system. As shown in FIG. 1, the ultrasound imaging system PA1 is electrically connected to an ultrasound probe PA2, the ultrasound probe PA2 includes M channels PA21 (only one is labelled).

The ultrasound imaging system PA1 includes a processing module PA11 and M analog-to-digital (A/D) modules PA12, wherein the processing module PA11 receives the echo signals PAS1 from the M channels PA21, transforms the echo signals PAS1 of the M channels PA21 into digital signals by using the A/D modules PA12 (the number of the channels PA21 is identical to the number of the A/D modules PA12, that is, if M is 128, the number of the A/D modules PA12 would be also 128), and proceeds linear and non-linear delay calculations to the digitalized echo signals and then accumulates the calculated results. The amount of delay can be represented as −(xi*sin θ/c)+(((xi)²*cos² θ)/(2*R*c)), wherein the part −(xi*sin θ/c) represents linear steering delay, and the part (((xi)²*cos² θ)/(2*R*c)) represents non-linear focusing delay, wherein xi is the distance between the channel of the ultrasound probe PA2 and the central channel, R is the distance between the object to be detected and the central channel of the ultrasound probe PA2, θ is the angle between R and the central channel of the ultrasound probe PA2, and c is the sound wave speed.

The circuit design as mentioned above has the potential to achieve better image imaging quality, however, because the number of the A/D modules PA12 should be identical to the number of the channels PA21, the increasing number of channels may result in high cost and high complexity of circuit design of the ultrasound imaging system PA1, and a significant space may be needed for locating the circuit of the ultrasound imaging system PA1. FIG. 2 is a block diagram of a second conventional ultrasound imaging system. As shown, the ultrasound imaging system PA1a is also electrically connected to an ultrasound probe PA2a, the ultrasound probe PA2a includes M channels PA21a (only one of them is labelled) divided into m groups in general, and each group includes n channels (i.e. m*n=M).

The ultrasound imaging system PA1a includes a processing module PA11a and m A/D modules PA12a, wherein the processing module PA11a receives the signals from the M channels and divides the signals into m groups, and each group includes the echo signals PAS1a received by n channels. The linear steering delays are applied to the signals and then the delayed signals are summed and transmitted to the m A/D modules PA12a (the number of the A/D modules is identical to the number of the groups, that is, if M is 128, m is 32, and n is 4, the number of the A/D modules would be 32). Thereafter, the non-linear focusing delays are applied to the pre-summed signals and then accumulating calculations are applied to the signals in the digital system to reconstruct the image.

The circuit design as mentioned above can reduce the number of A/D modules PA12a effectively, but will have the problem of degrading system resolution and image quality.

### SUMMARY OF THE INVENTION

In view of the conventional ultrasound imaging system, it is common to have the problem regarding tradeoff between the number of channels (each channel accompanies one A/D module and system complexity increases as the channel counts increased image quality. Accordingly, an image compensation system and a method thereof is provided in the present invention, which mainly features the technology of compensating the echo signal by using the digitalized plus-or-minus operators, arithmetic mean, and average signal strength value to achieve the object of reducing the number of A/D modules with acceptable image quality

According to the above mentioned object, an image compensation system is provided in accordance with an embodiment of the present invention. The image compensation system is electrically connected to an ultrasound probe for compensating a plurality of echo signals received by the ultrasound probe. The ultrasound probe includes M channels divided into m groups, and each of the groups includes n channels. The image compensation system comprises an average value calculation module, an error value calculation module, an average error value calculation module, K first analog-to-digital (A/D) modules, m second A/D modules corresponding to the m groups, m third A/D modules corresponding to the m groups, and a processing module. The average value calculation module is electrically connected to the ultrasound probe for receiving the echo signals from the n channels of each of the groups to generate n signal strengths corresponding to the n echo signals at a parsing time, accumulating the n signal strengths to generate an accumulated signal strength, and dividing the accumulated signal strength by n to generate an average signal strength value corresponding to each of the groups.

The error value calculation module is electrically connected to the average value calculation module for receiving the average signal strength value of each of the groups and the n signal strengths to generate n error values, and deciding n plus-or-minus operators corresponding to each of the groups according to positive or negative corresponding to the n error values respectively. The average error value calculation module is electrically connected to the error value calculation module for receiving the n error values corresponding to each of the groups, generating n absolute error values corresponding to the n error values respectively, calculating an arithmetic mean of the n absolute error value, and defining the arithmetic mean as an average absolute error value corresponding to each of the groups. Each of the first A/D modules includes N pins and is electrically connected to the error value calculation module for receiving the n plus-or-minus operators to transform the n plus-or-minus operators into n digitalized plus-or-minus operators respectively corresponding to each of the groups.

Each of the second A/D modules is electrically connected to the average error value calculation module for receiving the average absolute error value corresponding to each of the groups for transforming the average absolute error value into a digitalized average absolute error value corresponding to each of the groups. Each of the third A/D modules is electrically connected to the average value calculation module for receiving the average signal strength value corresponding to each of the groups for transforming the average signal strength value into a digitalized average signal strength value corresponding to each of the groups. The processing module is electrically connected to the K first A/D modules, the m second A/D modules, and the m third A/D modules, for calculating n compensated echo signals of each of the groups according to the n digitalized plus-or-minus operators, the digitalized average absolute error value and the digitalized average signal strength value. Wherein, K is an integer of rounding up M/N, and K+(m)+(m)<M.

In accordance with an embodiment of the present invention, the image compensation system further comprises a receiving module, which is electrically connected between the ultrasound probe and the average value calculation module, and is also electrically connected to the error value calculation module for receiving the n echo signals from the n channels of each of the groups at the parsing time. In addition, in accordance with an embodiment of the present invention, the average error value calculation module calculates the arithmetic mean by using minimum mean square error (MMSE) estimation, and the K first A/D modules, the m second A/D module, and the m third A/D modules are A/D converters. In addition, in accordance with an embodiment of the image compensation system of the present invention, each of the n plus-or-minus operators is a plus operator or a minus operator. As one of the n plus-or-minus operators is the plus operator, the digitalized plus-or-minus operator of the n digitalized plus-or-minus operators corresponding to the one of the n plus-or-minus operators corresponding to the plus operator is 1. As one of the n plus-or-minus operators is the minus operator, the digitalized plus-or-minus operator of the n digitalized plus-or-minus operators corresponding to the one of the n plus-or-minus operators corresponding to the minus operator is 0.

According to the above mentioned object, an image compensation method is also provided in accordance with an embodiment of the present invention. The image compensation method is applied to the above mentioned image compensation system and an ultrasound probe connected thereto, for compensating a plurality of echo signals received by the ultrasound probe. The ultrasound probe includes M channels divided into m groups, and each of the groups includes n channels. The image compensation method comprises steps (a) to (e). Step (a) is to receive the echo signals from the n channels of each of the groups to generate n signal strengths corresponding to the n echo signals at a parsing time, accumulate the n signal strengths to generate an accumulated signal strength, and divide the accumulated signal strength by n to generate an average signal strength value corresponding to each of the groups. Step (b) is to receive the average signal strength value of each of the groups and the n signal strengths to generate n error values, and decide n plus-or-minus operators corresponding to each of the groups according to positive or negative corresponding to the n error values respectively. Step (c) is to receive the n error values corresponding to each of the groups, generate n absolute error values corresponding to the n error values respectively, calculate an arithmetic mean of the n absolute error value, and define the arithmetic mean as an average absolute error value corresponding to each of the groups.

Step (d) is to receive the n plus-or-minus operators, the average absolute error value corresponding to each of the groups, and the average signal strength value corresponding to each of the groups, to transform the n plus-or-minus operators, the average absolute error value corresponding to each of the groups, and the average signal strength value corresponding to each of the groups into n digitalized plus-or-minus operators corresponding to each of the groups, a digitalized average absolute error value, and a digitalized average signal strength value. Step (e) is to calculate n compensated echo signals of each of the groups according to the n digitalized plus-or-minus operators, the digitalized average absolute error value, and the digitalized average signal strength value. Wherein, K is an integer of rounding up M/N, and K+(m)+(m)<M.

In accordance with an embodiment of the image compensation method of the present invention, each of the n plus-or-minus operators is a plus operator or a minus operator. As one of the n plus-or-minus operators is the plus operator, the digitalized plus-or-minus operator of the n digitalized plus-or-minus operators corresponding to the one of the n plus-or-minus operators corresponding to the plus operator is 1. As one of the n plus-or-minus operators is the minus operator, the digitalized plus-or-minus operator of the n digitalized plus-or-minus operators corresponding to the one of the n plus-or-minus operators corresponding to the minus operator is 0.

By using the image compensation system and the compensation method thereof provided in the embodiment of the present invention, the number of A/D modules can be significantly reduced because the echo signal is compensated by using the digitalized plus-or-minus operators, arithmetic mean, and average signal strength value, and the distorted image can be effectively compensated during image processing procedures, such that image quality can be effectively enhanced to facilitate practical usage.

### BRIEF DESCRIPTION OF THE DRAWINGS

The present invention will now be specified with reference to its preferred embodiment illustrated in the drawings, in which:

FIG. 1 is a block diagram of a first conventional ultrasound imaging system.

FIG. 2 is a block diagram of a second conventional ultrasound imaging system.

FIG. 3 is a block diagram of an image compensation system in accordance with a preferred embodiment of the present invention.

FIG. 4 is a flow chart showing an image compensation method in accordance with a preferred embodiment of the present invention.

FIG. 5 to FIG. 8 are schematic diagrams showing the waveforms of the echo signal in accordance with a preferred embodiment of the present invention.

FIG. 9 to FIG. 12 are schematic diagrams showing the waveforms of the compensated echo signal in accordance with a preferred embodiment of the present invention.

FIG. 13 is a schematic diagram showing the compensated echo signal of the second conventional ultrasound imaging system.

FIG. 14 shows a simulation image by using the first conventional ultrasound imaging system.

FIG. 15 shows a simulation image by using the second conventional ultrasound imaging system.

FIG. 16 shows a simulation image by using the image compensation system in accordance with the preferred embodiment of the present invention.

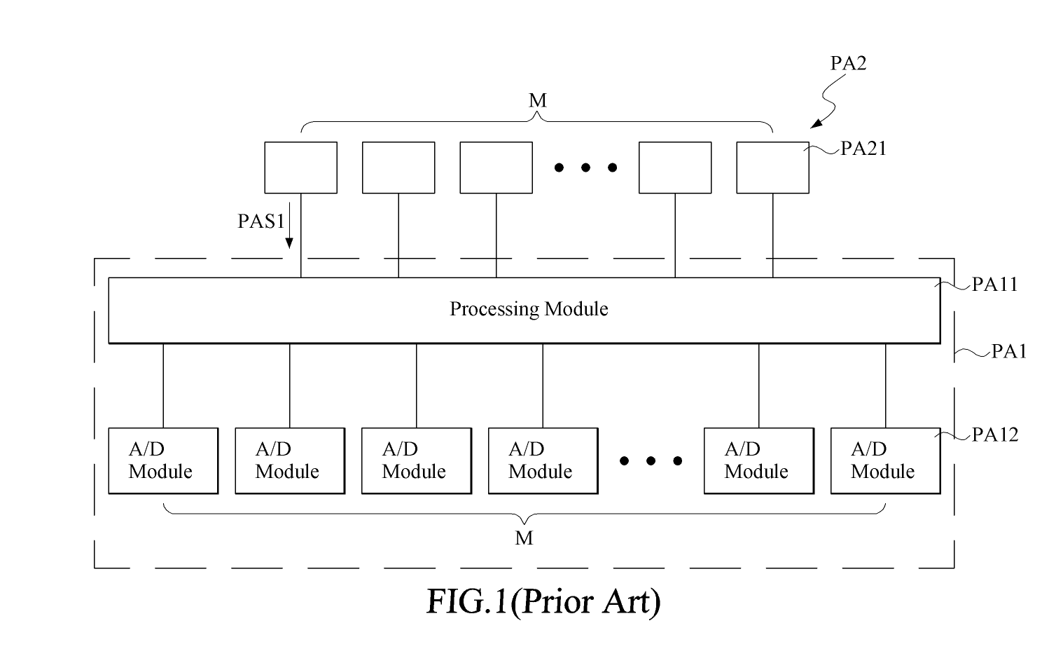

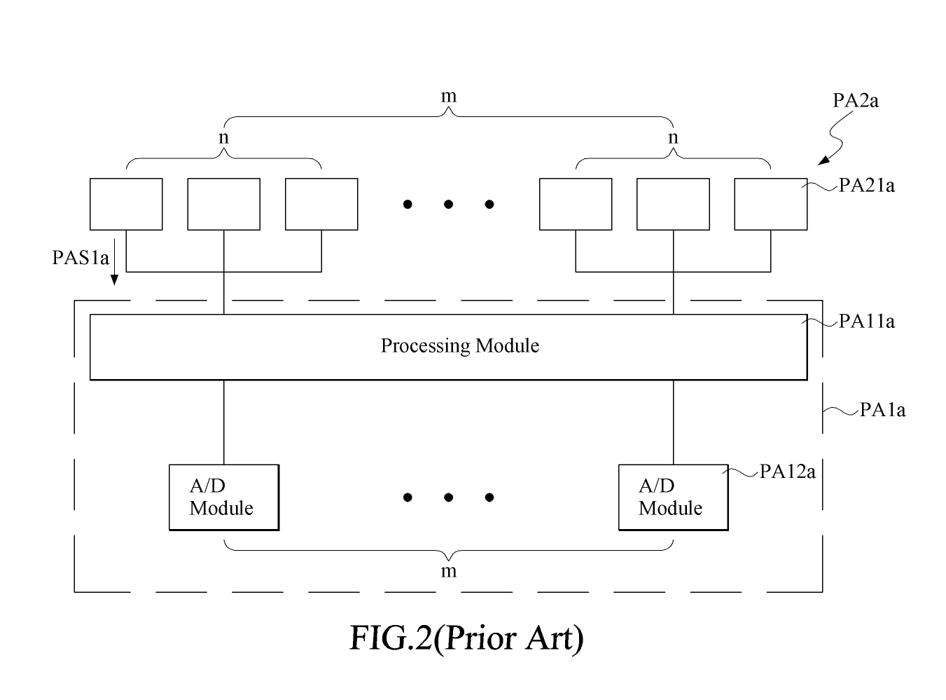

### DESCRIPTION OF THE PREFERRED EMBODIMENT

There are various embodiments of the image compensation system and the method thereof in accordance with the present invention, which are not repeated hereby. Only one preferred embodiment is mentioned in the following paragraph as an example.

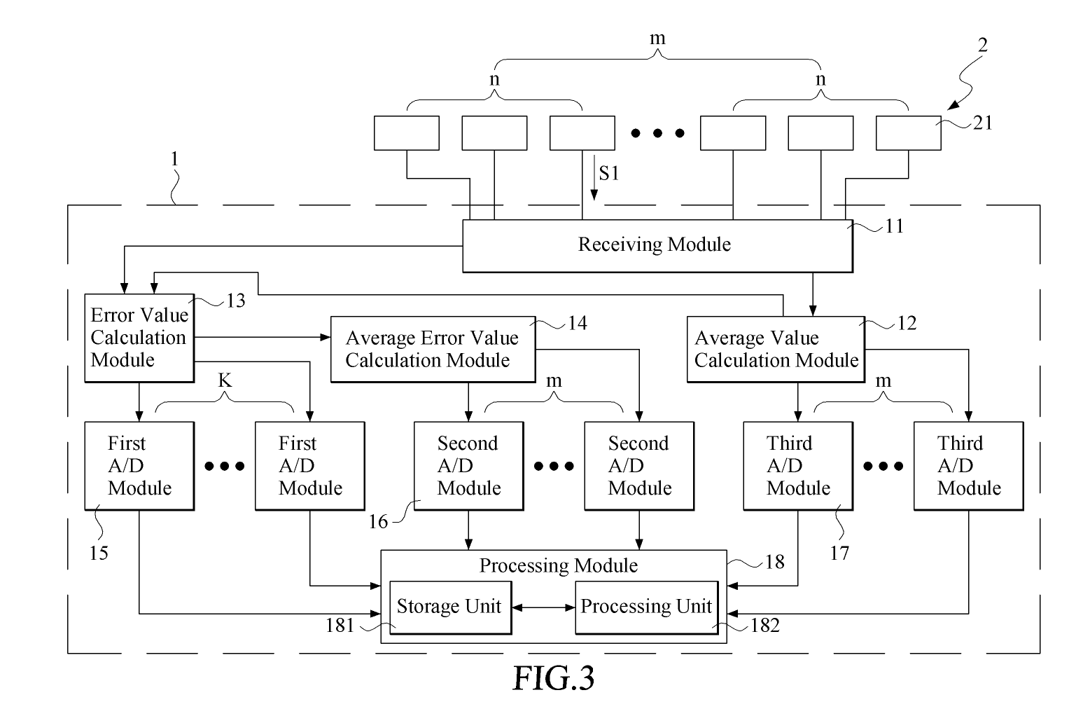

Please refer to FIG. 3, which is a block diagram of an image compensation system in accordance with a preferred embodiment of the present invention. As shown, the image compensation system 1 provided in accordance with the preferred embodiment of the present invention is electrically connected to an ultrasound probe 2 for compensating a plurality of echo signals S1 received by the ultrasound probe 2. The echo signal S1 is defined as the reflection signal of an ultrasonic signal (not shown in this figure) from the ultrasound probe 2 to an object to be detected (not shown in this figure). The ultrasound probe 2 includes M channels 21, the M channels 21 are divided into m groups, and each of the groups includes n channels 21 (only one of them is labelled in the figure), i.e. m*n=M. As a preferred embodiment of the present invention, the ultrasound probe 2 can be a one-dimensional (1D) probe or a two-dimensional (2D) probe, and the 2D probe is preferred in practice. As a preferred embodiment of the present invention, for example, M can be 128, m can be 32, and n can be 4, however, the present invention is not so restricted.

The image compensation system 1 comprises a receiving module 11, an average value calculation module 12, an error value calculation module 13, an average error value calculation module 14, K first analog-to-digital (A/D) modules 15 (only one of them is labelled in the figure), m second A/D modules 16 (only one of them is labelled) corresponding to the above mentioned m groups, m third A/D modules 17 (only one of them is labelled) corresponding to the above mentioned m groups, and a processing module 18.

The receiving module 11 is electrically connected to the ultrasound probe 2. In general, the receiving module 11 may be composed of the typical ultrasound probe processing circuit. The average value calculation module 12 is electrically connected to the receiving module 11 and can be composed of the typical operational amplifier (such as an averaging circuit) and some other components, however, the present invention is not so restricted. The error value calculation module 13 is electrically connected to the receiving module 11 and the average value calculation module 12 and can be composed of the subtractor, the comparator, etc., however, the present invention is not so restricted. The average error value calculation module 14 is electrically connected to the error value calculation module 13 and can be composed of the full-wave rectifier and the operational amplifier, however, the present invention is not so restricted.

Each first A/D module 15 includes N pins (for example, N is 16 in the preferred embodiment of the present invention) and is electrically connected to the error value calculation module 13. The second A/D module 16 is electrically connected to the average error value calculation module 14, the third A/D module 17 is electrically connected to the average value calculation module 12, and the above mentioned K first A/D module 15, the above mentioned m second A/D modules 16 and the above mentioned m third A/D modules 17 can be an analog-to-digital convertor.

The processing module 18 may be implemented by using the existed digital system to execute calculation of digital values. The processing module 18 may include a storage unit 181 and a processing unit 182, wherein the storage unit 181 can be a typical memory, and the processing module 182 electrically connected to the storage unit 181 can be a typical processor.

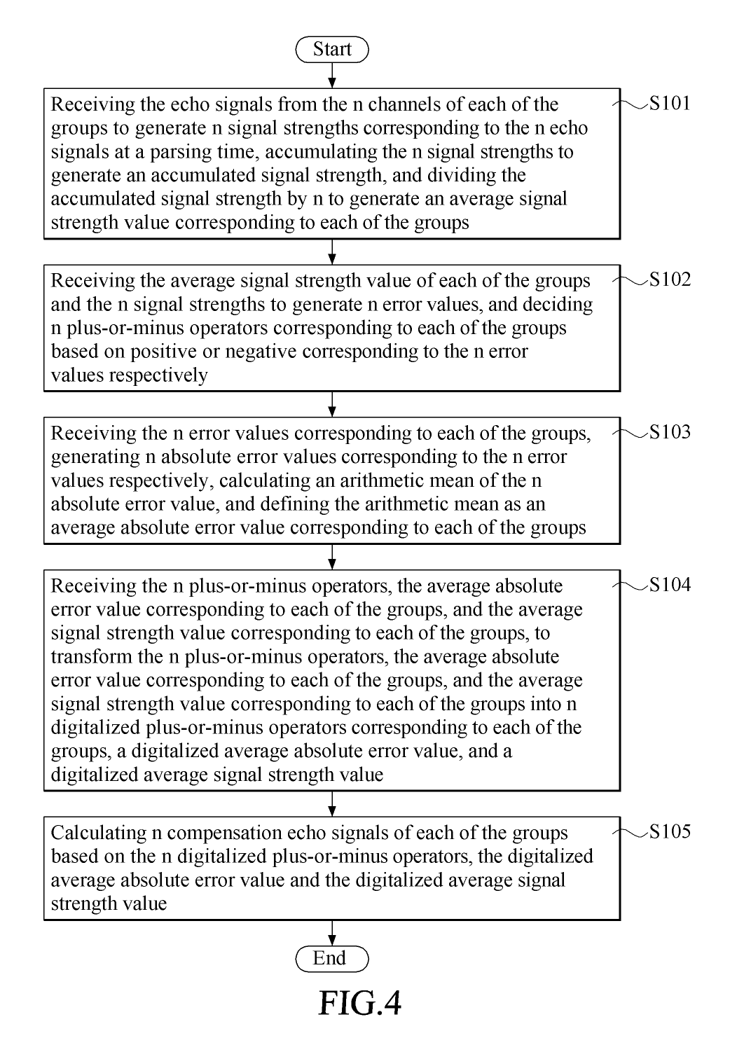

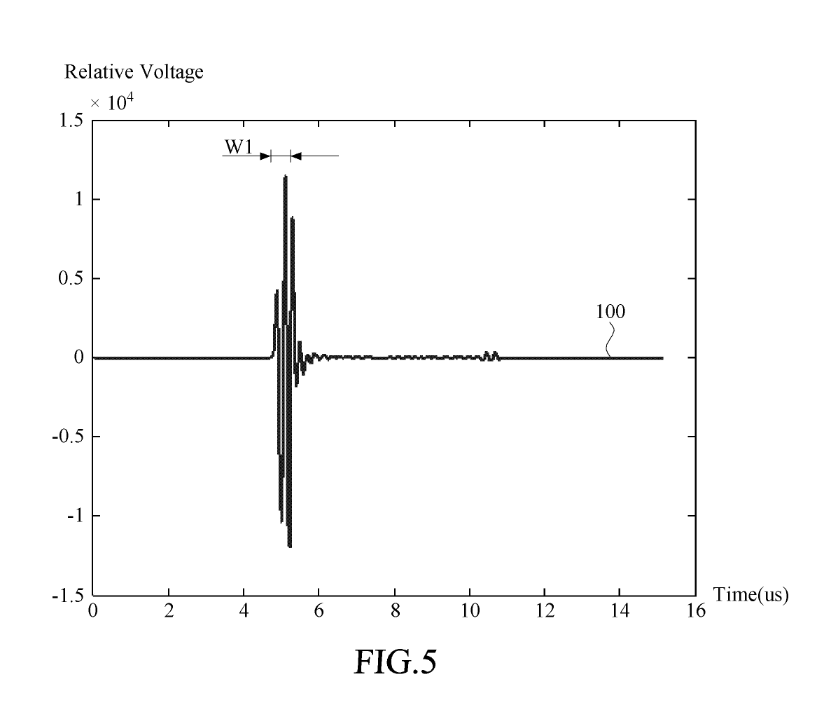

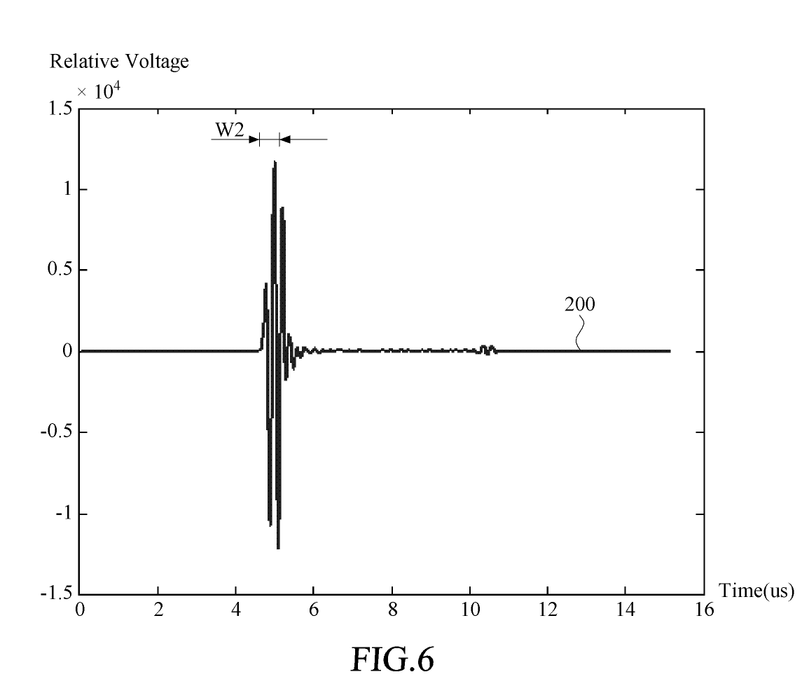

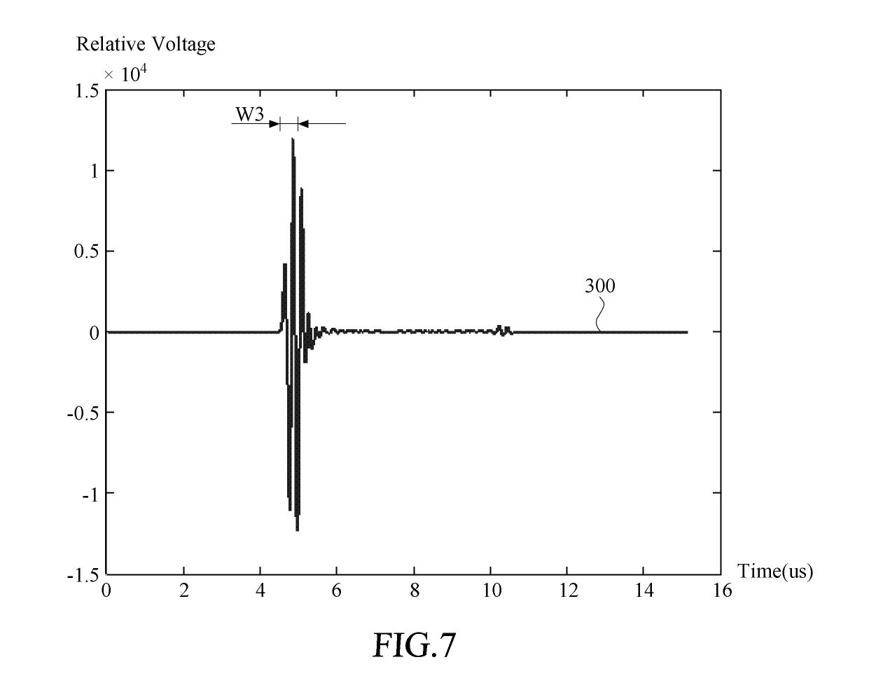

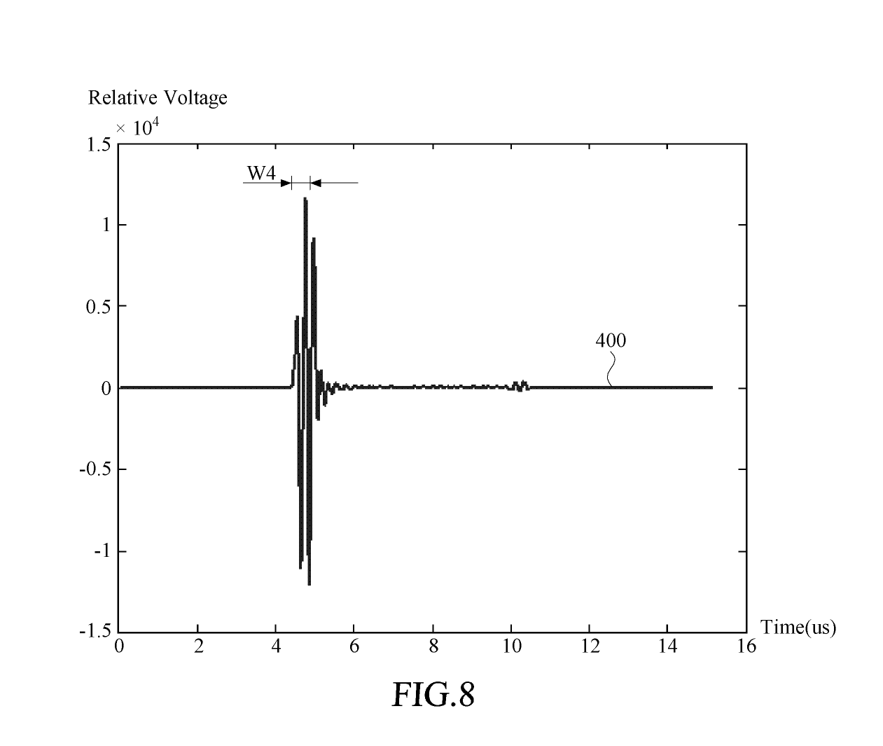

For a better understanding of the operation of the image compensation system 1, please refer to FIGS. 3 to 8, wherein FIG. 4 is a flow chart showing an image compensation method in accordance with a preferred embodiment of the present invention, and FIGS. 5 to 8 are schematic diagrams showing the waveforms of the echo signal in accordance with a preferred embodiment of the present invention. As shown, the image compensation method comprises the following steps:

Step S101 is to receive the echo signals from the n channels of each of the groups to generate n signal strengths corresponding to the n echo signals at a parsing time, accumulate the n signal strengths to generate an accumulated signal strength, and divide the accumulated signal strength by n to generate an average signal strength value corresponding to each of the groups.

Step S102 is to receive the average signal strength value of each of the groups and the n signal strengths to generate n error values, and decide n plus-or-minus operators corresponding to each of the groups according to positive or negative corresponding to the n error values respectively.

Step S103 is to receive the n error values corresponding to each of the groups, generate n absolute error values corresponding to the n error values respectively, calculate an arithmetic mean of the n absolute error value, and define the arithmetic mean as an average absolute error value corresponding to each of the groups.

Step S104 is to receive the n plus-or-minus operators, the average absolute error value corresponding to each of the groups, and the average signal strength value corresponding to each of the groups, to transform the n plus-or-minus operators, the average absolute error value corresponding to each of the groups, and the average signal strength value corresponding to each of the groups into n digitalized plus-or-minus operators corresponding to each of the groups, a digitalized average absolute error value, and a digitalized average signal strength value.

Step S105 is to calculate n compensated echo signals of each of the groups according to the n digitalized plus-or-minus operators, the digitalized average absolute error value and the digitalized average signal strength value.

In Step S101, the receiving module 11 is utilized for receiving the n echo signals of the plurality of echo signals S1 from the n channels 21 at a parsing time. It should be noted that these echo signals S1 can be the steered signals (or the un-steered ones which would be steered in the receiving module 11), such as those being applied with delaying operation. The parsing time can be 0 microsecond, 2 microsecond, 4 microsecond, and so on to 16 microsecond shown in FIG. 5 to FIG. 8 (it is preferred to use the parsing time second decimal digitals as the unit, such as 0.12 microsecond, thus, steps S101 to S105 of the present invention is for dealing with the echo signals of different parsing time), however, the present invention is not so restricted.

In addition, the receiving module 11 receives the above mentioned n echo signals S1 to the average value calculation module 12. The average value calculation module 12 receives the n echo signals 21 of these echo signals S1 from the n channels 21 to generate n signal strengths corresponding to the n echo signals at the parsing time, accumulates the n signal strengths to generate an accumulated signal strength, and divides the accumulated signal strength by n to generate an average signal strength value corresponding to each of the groups. As a preferred embodiment, the step S101 can be implemented by using the averaging circuit.

For example, as a preferred embodiment of the present invention, if n is 4, there would be four echo signals S1 (represented by waveforms 100, 200, 300, and 400 in FIGS. 5 to 8 respectively, and it should be noted that the initial time of the first wave corresponding to the widths W1, W2, W3, and W4 is 4.68 microsecond, the end time of the fourth wave is 5.64 microsecond, and thus the widths W1, W2, W3, and W4 are 0.96 microsecond).

The average value calculation module 12 generates four signals strengths (i.e. voltage value or current value of each point in the signal waveform, and the four signal strengths are represented as A, B, C, and D) after receiving the four echo signals S1. The average value calculation module 12 accumulates the signal strengths directly to access the accumulated signal strength (each point in the signal waveform has a corresponding accumulated signal strength), and then divides the accumulated signal strength by 4 to access the average signal strength value (each point in the signal waveform has a corresponding average signal strength value, which is represented as DS).

In step S102, the error value calculation module 13 receives the average signal strength value DS corresponding to each of the groups and the above mentioned n signal strengths A, B, C, and D to generate n error values (represented as Da, Db, Dc, and Dd in the following paragraphs), and decides n plus-or-minus operators corresponding to each of the groups according to positive or negative corresponding to the n error values respectively. Each of the aforementioned plus-or-minus operator is a plus operator or a minus operator (which may be implemented by using a comparator for example).

For example, in accordance with the preferred embodiment of the present invention, in which n is 4, the error value calculation module 13 may calculate the error values Da, Db, Dc, and Dd using the equations Da=DS-A, Db=DS-B, Dc=DS-C, and Dd=DS-D. Preferably, the error value calculation module 13 may use the subtractors to execute the above mentioned calculations. Then, the four plus-or-minus operators corresponding to each of the groups are decided according to positive or negative of the above mentioned error values Da, Db, Dc, and Dd. For example, if the error value Da is positive, the error value Db is positive, the error value Dc is negative, and the error value Dd is negative, then the corresponding plus-or-minus operators would be plus, plus, minus, and minus respectively.

In step S103, the average error value calculation module 14 receives the n error values corresponding to each of the groups, and generates n absolute error values corresponding to the n error values respectively. Preferably, the n absolute error value can be generated by using an absolute value calculator. After completing the above mentioned steps, an arithmetic mean of the n absolute error value may be further calculated and defined as an average absolute error value corresponding to each of the groups. The arithmetic mean can be calculated by using an average calculator.

For example, the average error value calculation module 14 receives the four error values Da, Db, Dc, and Dd, calculates four corresponding absolute error values |Da|, |Db|, |Dc| and |Dd| by using the full-wave rectifier, and then calculates the arithmetic mean (which may be implemented by using the operational amplifier), e.g. (|Da|+|Db|+|Dc|+|Dd|)/4, which is defined as an average absolute error value (represented as ED in the following paragraphs) corresponding to each of the groups.

In step S104, each of the first A/D modules 15 receives the above mentioned n plus-or-minus operators to transform the n plus-or-minus operators into n digitalized plus-or-minus operators respectively corresponding to each of the groups. As one of the n plus-or-minus operators is the plus operator, the digitalized plus-or-minus operator of the n digitalized plus-or-minus operators corresponding to the one of the n plus-or-minus operators corresponding to the plus operator is 1. However, the present invention is not so restricted. As one of the n plus-or-minus operators is the minus operator, the digitalized plus-or-minus operator of the n digitalized plus-or-minus operators corresponding to the one of the n plus-or-minus operators corresponding to the minus operator is 0. However, the present invention is not so restricted. As for the present preferred embodiment, in which n is 4, the four digitalized plus-or-minus operators would be 1, 1, 0, and 0.

Each of the second A/D modules 16 receives the average absolute error value corresponding to each of the groups for transforming the average absolute error value ED into a digitalized average absolute error value (represented as a in the following paragraphs) corresponding to each of the groups. Each of the third A/D modules 17 receives the average signal strength value corresponding to each of the groups for transforming the average signal strength value into a digitalized average signal strength value (represented as b in the following paragraphs) corresponding to each of the groups. The above mentioned digitalized processing may reduce distortion during signal processing so as to reduce distortion in the following image compensation step.

In step S105, the storage unit 181 of the processing module 18 stores the n digitalized plus-or-minus operators corresponding to each of the groups, the digitalized average absolute error value corresponding to each of the groups, and the digitalized average signal strength value corresponding to each of the groups.

The processing unit 182 fetches the n digitalized plus-or-minus operators corresponding to each of the groups, the digitalized average absolute error value corresponding to each of the groups and the digitalized average signal strength value corresponding to each of the groups in the storage unit 181, and calculates n compensated echo signals (represented as A′, B′, C′ and D′ in the following paragraphs) for each of the groups according to the n digitalized plus-or-minus operators, the digitalized average absolute error value, and the digitalized average signal strength value. As for the present preferred embodiment in which n is 4, the compensated echo signal A′ can be b+a, the compensated echo signal B′ can be b+a, the compensation signal C′ can be b−a, and the compensated echo signal D′ can be b−a. However, the present invention is not so restricted. The number n can be any integer according to the need.

In addition, it should be noted that although the image compensation system 1 includes K first A/D modules 15, m second A/D modules 16, and m third A/D modules 17, when being applied to the ultrasound probe 2 used in the preferred embodiment of the present invention which includes M channels 21 divided into m groups and each group has n channels 21, it is required to satisfy the limitation that K is an integer of rounding up M/N (e.g. if M/N=7.1, then K=8), and K+(m)+(m)<M.

For example, in accordance with the preferred embodiment of the present invention, M is 128, m is 32, and N is 16, therefore K (K=M/N) would be 8. The number of A/D modules being used in the present invention would be 72 (i.e. K+(m)+(m)=72), which is smaller than M. Hence, in compared with the first conventional technology, the present invention has the potential to reduce the number of A/D modules effectively.

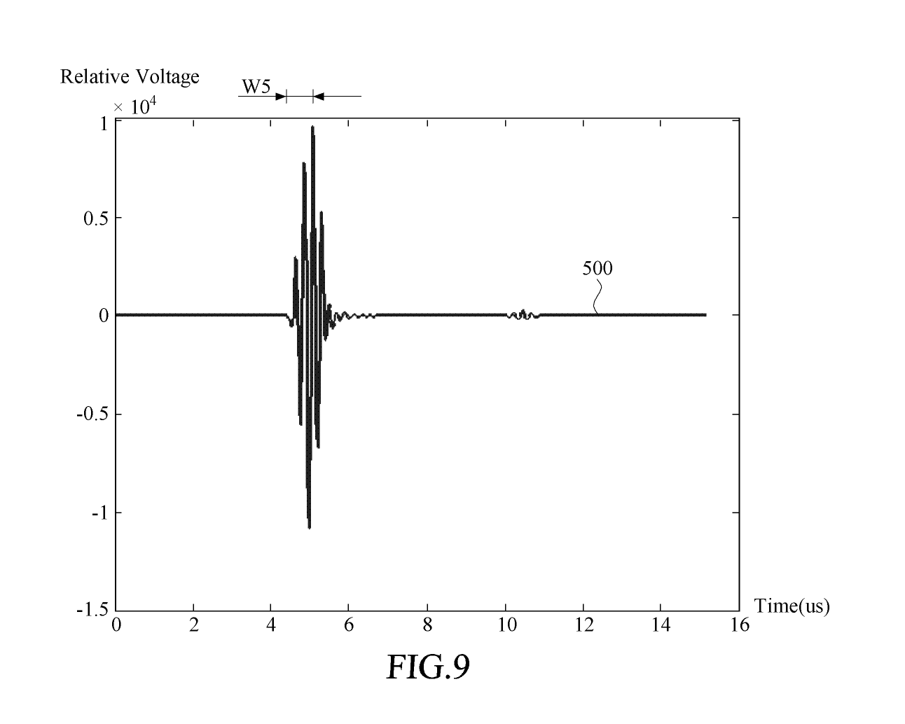

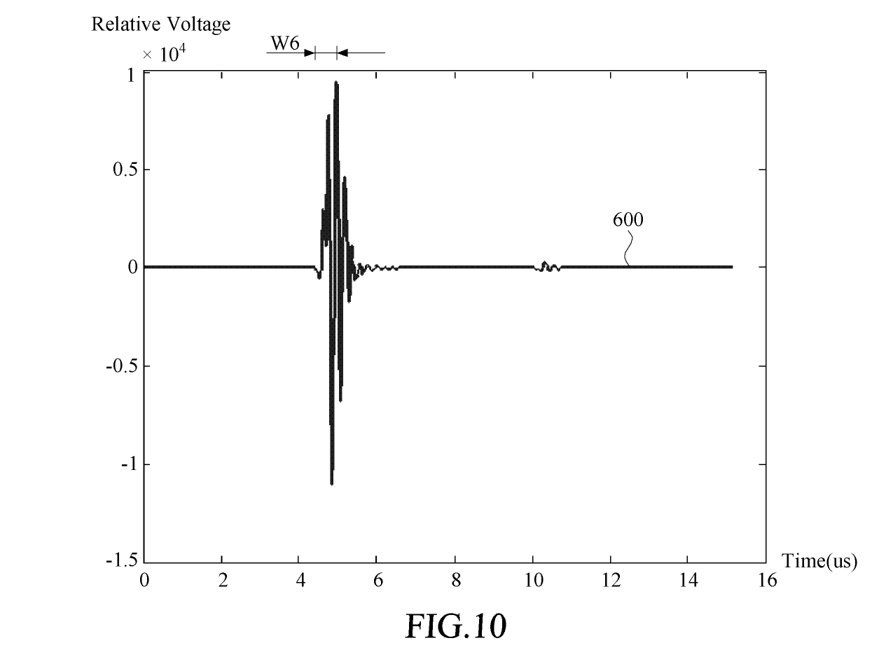

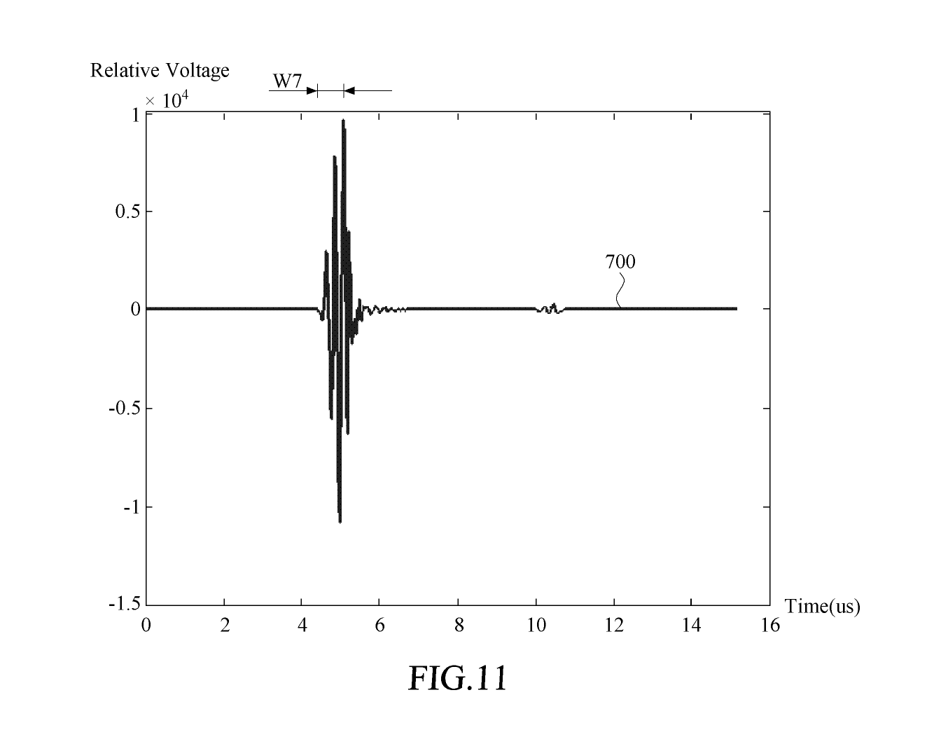

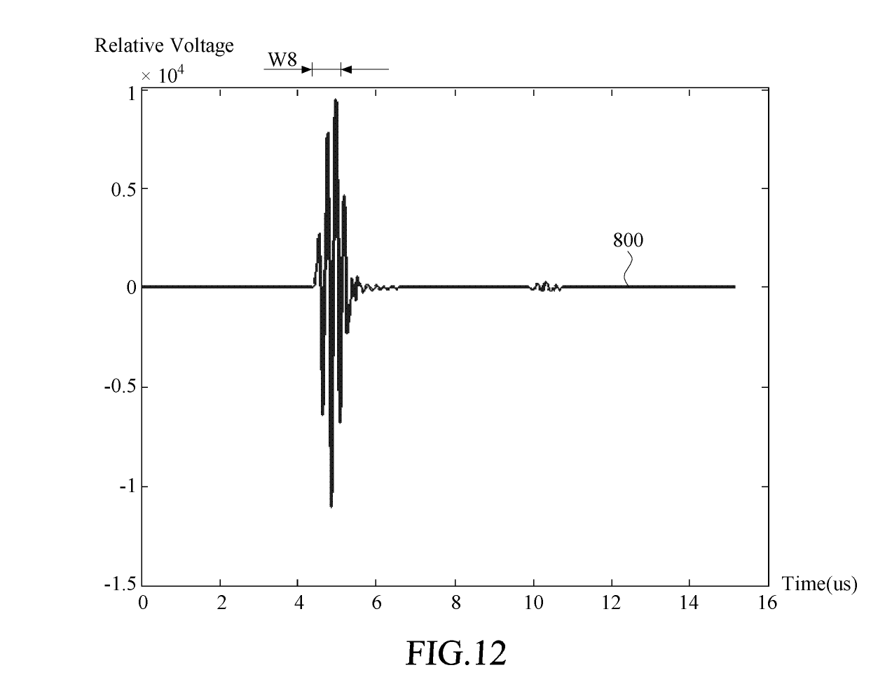

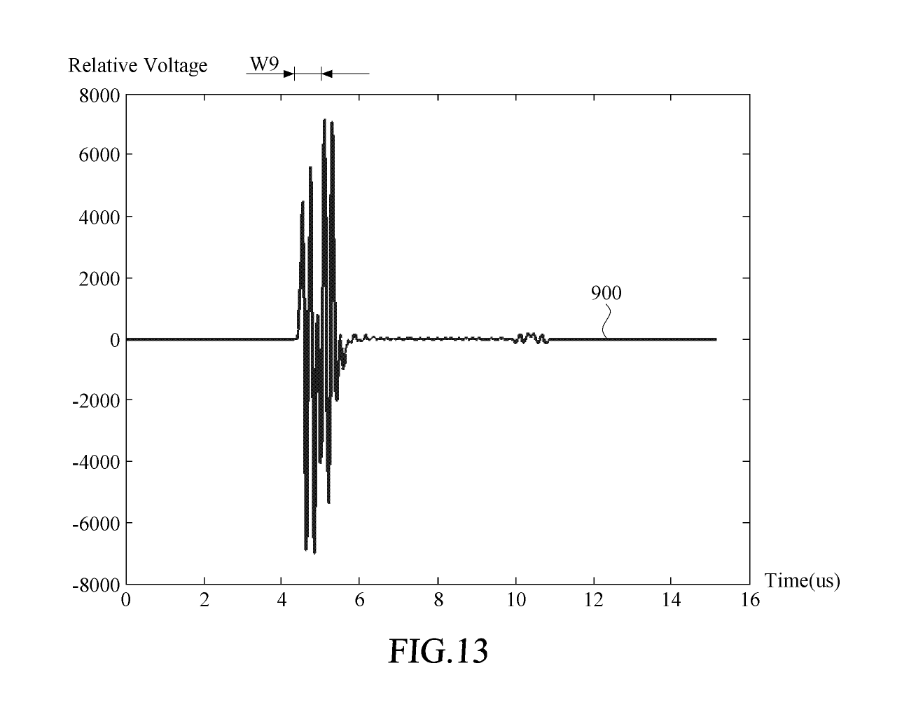

Please refer to FIGS. 9 to 13, wherein FIGS. 9 to 12 are schematic diagrams showing the waveforms of the compensated echo signal in accordance with a preferred embodiment of the present invention, and FIG. 13 is a schematic diagram showing the compensated echo signal of the second conventional ultrasound imaging system. As shown, the compensated simulation result of each of the parsing time after the calculations of steps S101 to S105 would generate the waveforms 500, 600, 700, and 800 corresponding to the four echo signals S1 (waveforms 100, 200, 300, and 400) respectively. The initial time of the first wave of the width W5, W6, W7, and W8 is 4.58 microsecond, the end time of the sixth wave is 5.74, and thus the widths W5, W6, W7, and W8 are 1.16 microseconds.

The waveforms generated by using the first conventional technology are similar to the waveforms 100, 200, 300, and 400 (the term “similar” described in the preferred embodiment of the present invention indicates that the calculated result is within the acceptable error margin), and the waveforms 500, 600, 700, and 800 generated by using the image compensation system and the compensation method thereof provided in the present invention are similar to the waveforms 100, 200, 300, and 400, and the widths W5, W6, W7, and W8 thereof are not much different from the widths W1, W2, W3, and W4, therefore, in compared with the first conventional technology, which needs a great number of A/D modules, the present invention needs fewer A/D modules but is able to maintain the image quality to the level close to the first conventional technology.

The waveform 900 shown in FIG. 13 is the compensated result after the calculation of the second conventional technology, wherein the initial time of the first wave of the width W9 is 4.42 microsecond, the end time of the sixth wave is 5.82 microsecond, and thus the width is 1.4 microsecond, which is much longer than that of the preferred embodiment of the present invention, i.e. 1.16 microsecond. Thus it can be seen that the second conventional technology has the problems of a significant amount of noise and poor resolution although it uses fewer A/D modules. In contrast, the technology of the present invention applies the relation K+(m)+(m)<M to define an adequate number of A/D modules such that the problem of poor resolution can be properly resolved.

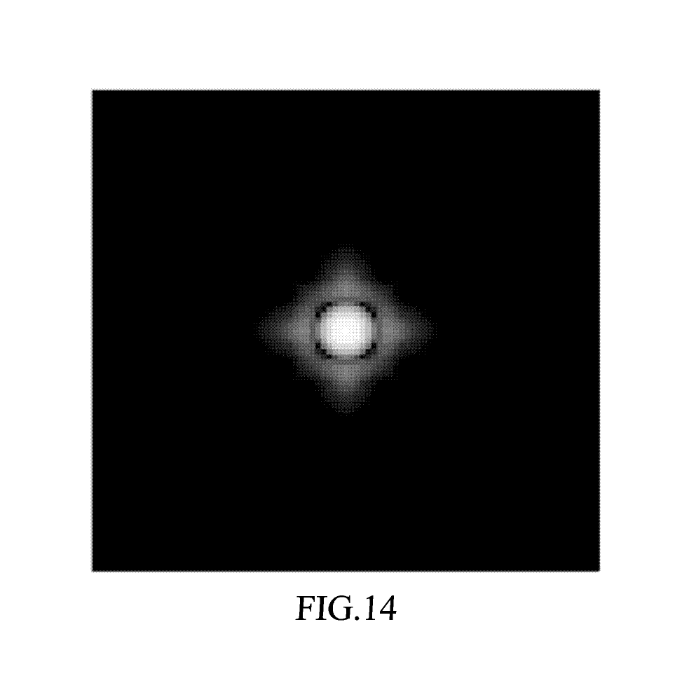

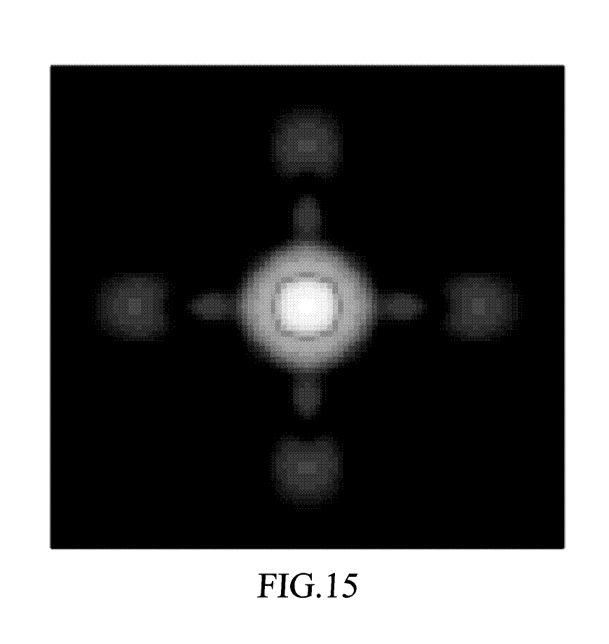

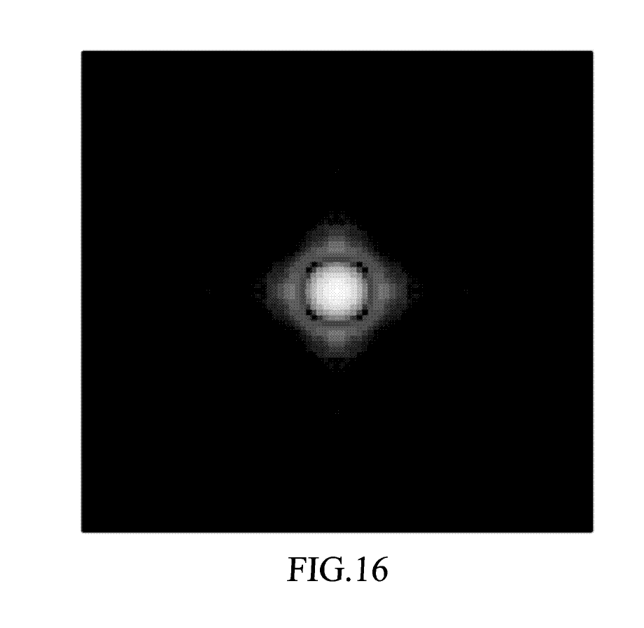

Please refer to FIGS. 14 to 16, wherein FIG. 14 shows a simulation image by using the first conventional ultrasound imaging system, FIG. 15 shows a simulation image by using the second conventional ultrasound imaging system, and FIG. 16 shows a simulation image by using the image compensation system provided in accordance with the preferred embodiment of the present invention. As shown, the image resolution using the present invention is close to that using the first conventional technology but much better than that using the second conventional technology. Thus, by using the technology provided in the present invention, the number of A/D modules can be significantly reduced in compared with the first conventional technology while maintaining acceptable image resolution. In addition, although only the 1D ultrasound probe is described in the present invention, but the present invention is not so restricted, the technology described in the preferred embodiment of the present invention should be able to be applied to the case with 2D ultrasound probe or more.

In conclusion, by using the image compensation system and the compensation method thereof provided in the embodiment of the present invention, the number of A/D modules can be significantly reduced because the echo signal is compensated by using the digitalized plus-or-minus operators, arithmetic mean, and average signal strength value, and the distorted image can be effectively compensated during image processing procedures, such that image quality can be effectively enhanced to facilitate the practical usage.

The detail description of the above mentioned preferred embodiments is for clarifying the feature and the spirit of the present invention. The present invention should not be limited by any of the exemplary embodiments described herein, but should be defined only in accordance with the following claims and their equivalents. Specifically, those skilled in the art should appreciate that they can readily use the disclosed conception and specific embodiments as a basis for designing or modifying other structures for carrying out the same purposes of the present invention without departing from the scope of the invention as defined by the appended claims.

1. An image compensation system, for electrically connecting to an ultrasound probe for compensating a plurality of echo signals received by the ultrasound probe, wherein the ultrasound probe includes M channels divided into m groups, and each of the groups includes n channels, and the image compensation system comprising:
an average value calculation module, electrically connected to the ultrasound probe, for receiving the echo signals from the n channels of each of the groups to generate n signal strengths corresponding to the n echo signals at a parsing time, accumulating the n signal strengths to generate an accumulated signal strength, and dividing the accumulated signal strength by n to generate an average signal strength value corresponding to each of the groups, wherein the average value calculation module comprises an operational amplifier;
an error value calculation module, electrically connected to the average value calculation module, for receiving the average signal strength value of each of the groups and the n signal strengths to generate n error values, and assigning n plus-or-minus operators corresponding to each of the groups according to positive or negative symbol corresponding to the n error values respectively, wherein the error value calculation module comprises a subtractor and a comparator;
an average error value calculation module, electrically connected to the error value calculation module, for receiving the n error values corresponding to each of the groups, generating n absolute error values corresponding to the n error values respectively, and calculating an arithmetic mean of the n absolute error value and defining the arithmetic mean as an average absolute error value corresponding to each of the groups, wherein the average error value calculation module comprises a rectifier and an operational amplifier;
K first analog-to-digital (A/D) modules, each of the first A/D modules including N pins and electrically connected to the error value calculation module for receiving the n plus-or-minus operators and transforming the n plus-or-minus operators into n digitalized plus-or-minus operators respectively corresponding to each of the groups;
m second A/D modules corresponding to the m groups, electrically connected to the average error value calculation module for receiving the average absolute error value corresponding to each of the groups and transforming the average absolute error value into a digitalized average absolute error value corresponding to each of the groups;
m third A/D modules corresponding to the m groups, electrically connected to the average value calculation module, for receiving the average signal strength value corresponding to each of the groups and transforming the average signal strength value into a digitalized average signal strength value corresponding to each of the groups; and
a processing module, electrically connected to the K first A/D modules, the m second A/D modules, and the m third A/D modules, for calculating n compensated echo signals of each of the groups according to the n digitalized plus-or-minus operators, the digitalized average absolute error value and the digitalized average signal strength value;
wherein, K is an integer of rounding up M/N, and K+(m)+(m)<M.

2. The image compensation system of claim 1, further comprising a receiving module, which is electrically connected to the average value calculation module, and is also electrically connected to the error value calculation module, for receiving the n echo signals from the n channels of each of the groups at the parsing time.

3. The image compensation system of claim 1, wherein the K first A/D modules, the m second A/D modules, and the m third A/D modules are A/D converters.

4. The image compensation system of claim 1, wherein each of the n plus-or-minus operators is a plus operator or a minus operator.

5. The image compensation system of claim 4, wherein the digitalized plus-or-minus operator corresponding to the plus operator is a digital signal 1; and the digitalized plus-or-minus operator corresponding to the minus operator is a digital signal 0.

6. An image compensation method, using the image compensation system of claim 1 and an ultrasound probe connected thereto, for compensating a plurality of echo signals received by the ultrasound probe, wherein the ultrasound probe includes M channels divided into m groups, and each of the groups includes n channels, and the image compensation method comprising:
(a) using an average value calculation module for, receiving the echo signals from the n channels of each of the groups to generate n signal strengths corresponding to the n echo signals at a parsing time, accumulating the n signal strengths to generate an accumulated signal strength, and dividing the accumulated signal strength by n to generate an average signal strength value corresponding to each of the groups;
(b) using an error value calculation module for, receiving the average signal strength value of each of the groups and the n signal strengths to generate n error values, and assigning n plus-or-minus operators corresponding to each of the groups according to positive or negative symbol corresponding to the n error values respectively;
(c) using an average error value calculation module for, receiving the n error values corresponding to each of the groups, generate n absolute error values corresponding to the n error values respectively, calculating an arithmetic mean of the n absolute error value, and defining the arithmetic mean as an average absolute error value corresponding to each of the groups;
(d) using K first analog-to-digital (A/D) modules for receiving the n plus-or-minus operators and transforming the n plus-or-minus operators into n digitalized plus-or-minus operators corresponding to each of the groups, using m second A/D modules corresponding to the m groups for receiving the average absolute error value corresponding to each of the groups and transforming the average absolute error value into a digitalized average absolute error value corresponding to each of the groups, and using m third A/D modules corresponding to the m groups for receiving the average signal strength value corresponding to each of the groups and transforming the average signal strength value into a digitalized average signal strength value corresponding to each of the groups; and
(e) using a processing module for calculating n compensated echo signals of each of the groups according to the n digitalized plus-or-minus operators, the digitalized average absolute error value, and the digitalized average signal strength value;
wherein, K is an integer of rounding up M/N, and K+(m)+(m)<M.

7. The image compensation method of claim 6, wherein each of the n plus-or-minus operators is a plus operator or a minus operator.

8. The image compensation method of claim 7, wherein a the digitalized plus-or-minus operator corresponding to the plus operator is a digital signal 1; and the digitalized plus-or-minus operator corresponding to the minus operator is a digital signal 0.
